(19) United States
(12) Patent Application Publication
(10) Pub. No.: US 2024/0390449 A1
(43) Pub. Date: Nov. 28, 2024

(54) COMPOSITION FOR INCREASING REGULATORY T CELLS

(71) Applicant: TOYO SHINYAKU CO., LTD., Fukuoka (JP)

(72) Inventors: Takumi MORIKAWA, Saga (JP); Seiichi KITAMURA, Saga (JP); Tokiyoshi AYABE, Hokkaido (JP); Kiminori NAKAMURA, Hokkaido (JP)

(73) Assignee: TOYO SHINYAKU CO., LTD., Fukuoka (JP)

(21) Appl. No.: 18/696,848

(22) PCT Filed: Aug. 9, 2022
(86) PCT No.: PCT/JP2022/030447

§ 371 Date: Mar. 28, 2024

(30) Foreign Application Priority Data
Sep. 30, 2021 (JP) 2021-160293

(51) Int. Cl. A61K 36/8998; A61P 37/06

(52) U.S. Cl. CPC A61K 36/8998; A61P 37/06

(57) ABSTRACT

It is to provide a composition comprising young barley grass, used for increasing regulatory T cells, suppressing immune response, maintaining normal immune function, and for anti-virus.

### TECHNICAL FIELD

The present invention relates to a composition that can increase regulatory T cells.

### BACKGROUND ART

Recently, against the background of the increase in interest on health, various health food products are developed, and various materials used in health food products are investigated.

Among these, young barley grass is used as material for health food products or green juice, and its functionality such as anti-cholesterol effect is widely investigated (for example, patent document 1).

### CITATION LIST

### Patent Literature

Patent document 1: Japanese Laid Open Patent Publication No. 2001-314170

### SUMMARY OF INVENTION

### Technical Problem

The object of the present invention is to provide a composition having a new function using young barley grass.

### Solution to Problem

The present inventors made a keen study on the functions of young barley grass, and they found out that young barley grass has an action of increasing regulatory T cells. The present invention has been completed.

Specifically, the present invention is as follows.
[1] A composition for increasing regulatory T cells, comprising young barley grass.
[2] A composition for suppressing immune response, comprising young barley grass.
[3] A composition for maintaining normal immune function, comprising young barley grass.
[4] An antiviral composition comprising young barley grass.
[5] A composition used to help to increase regulatory T cells, comprising young barley grass.
[6] A composition used to help to suppress immune response, comprising young barley grass.
[7] A composition used to help to maintain normal immune function, comprising young barley grass.
[8] A composition used to help to maintain or improve antiviral function, comprising young barley grass.
[9] A composition comprising young barley grass, added with an indication to be useful to increase regulatory T cells.
[10] A composition comprising young barley grass, added with an indication to be useful to suppress immune response.
[11] A composition comprising young barley grass, added with an indication to be useful to maintain normal immune function.
[12] A composition comprising young barley grass, added with an indication to be useful to maintain or improve antiviral function.
[13] A composition comprising young barley grass, added with an indication of one or more functions selected from “suppressing immune response”, “help to suppress immune response”, “useful to suppress immune response”, “maintain immune function to be normal”, “help to maintain immune function to be normal”, “useful to maintain immune function to be normal”, “maintain normal immune function”, “help to maintain normal immune function”, “useful to maintain normal immune function”, “maintain immunologic homeostasis”, “help to maintain immunologic homeostasis”, “useful to maintain immunologic homeostasis”, “maintain normal mucosal immune function”, “help to maintain normal mucosal immune function”, “useful to maintain normal immune function”, and “useful to maintain normal mucosal immune function”.
[14] A composition comprising young barley grass, added with an indication of one or more functions selected from “suppressing immune response”, “help to suppress immune response”, “useful to suppress immune response”, “maintain immune function to be normal”, “help to maintain immune function to be normal”, “useful to maintain immune function to be normal”, “maintain normal immune function”, “help to maintain normal immune function”, “useful to maintain normal immune function”, “maintain immunologic homeostasis”, “help to maintain immunologic homeostasis”, “useful to maintain immunologic homeostasis”, “maintain normal mucosal immune function”, “help to maintain normal mucosal immune function”, “useful to maintain normal immune function”, and “useful to maintain normal mucosal immune function”.
[15] A composition comprising young barley grass, added with an indication of one or more functions selected from “maintain barrier function of the body”, “help to maintain barrier function of the body”, “elevating barrier function of the body”, “help to elevate barrier function of the body”, “increasing barrier function of the body”, “help to increase barrier function of the body”, “enhancing barrier function of the body”, “help to enhance barrier function of the body”, “maintain protection of the body”, “hep to maintain protection of the body”, “elevating protection of the body”, “hep to elevate protection of the body”, “maintain barrier function of mucosa”, “help to maintain barrier function of mucosa”, “elevate barrier function of mucosa”, “help to elevate protection of mucosa”, “maintain protection of mucosa”, “help to maintain protection of mucosa”, “elevate protection of mucosa”, “help to elevate protection of mucosa”, “maintain protection against virus”, “help to maintain protection against virus”, “elevate protection against virus”, “help to elevate protection against virus”, “enhancing protection against virus”, “help to enhance protection against virus”, “maintain normal immune function”, and “help to maintain normal immune function”.
[16] Use of young barley grass for manufacturing a composition for increasing regulatory T cells.
[17] Use of young barley grass for manufacturing a composition for suppressing immune response.
[18] Use of young barley grass for manufacturing a composition for maintaining normal immune function.
[19] Use of young barley grass for manufacturing an antiviral composition.
[20] A method for increasing regulatory T cells, comprising administering a composition comprising young barley grass to human.
[21] A method for suppressing immune response, comprising administering a composition comprising young barley grass to human.
[22] A method for maintaining normal immune function, comprising administering a composition comprising young barley grass to human.
[23] A method for maintaining or improving antiviral function, comprising administering a composition comprising young barley grass to human.

### Advantageous Effects of Invention

Since the composition of the present invention can increase regulatory T cells, it is possible to suppress excessive proliferation, differentiation of immune cells such as effector T cells, etc. and to suppress immune response, to maintain normal immune function, and to suppress autoimmune diseases, allergic diseases (pollen allergy, food allergy, etc.), inflammatory diseases, etc. induced by excessive immune response.

### BRIEF DESCRIPTION OF DRAWING

FIG. 1 shows FoxP3 positive T cell ratio (Treg ratio) in the total CD4 positive cells in the Peyer's patch of mouse small intestine administered with young barley grass.

### DESCRIPTION OF EMBODIMENTS

The composition of the present invention is characterized by comprising young barley grass.

The composition of the present invention can increase regulatory T cells. Therefore, the composition of the present invention can be used as a composition for increasing regulatory T cells. Here, regulatory T cells in the present invention refer to Treg cells.

Since the composition of the present invention can increase regulatory T cells, it is possible to suppress excessive proliferation, differentiation of immune cells such as effector T cells, etc., to suppress immune response, and to maintain normal immune function. Therefore, it is possible to suppress autoimmune diseases, allergic diseases (pollen allergy, food allergy, etc.), inflammatory diseases, etc. induced by excessive immune response.

Specifically, the composition of the present invention can be used as a composition for suppressing immune response, a composition for maintaining normal immune function (composition for maintaining immunologic homeostasis), a composition for suppressing autoimmune diseases, a composition for suppressing allergic diseases, and a composition for suppressing inflammatory diseases. Here, to maintain normal immune function (maintain immunologic homeostasis) includes to maintain normal mucosal immune function.

Further, since the composition of the present invention can increase regulatory T cells, it is possible to maintain or improve immune function. Therefore, it is possible to maintain or improve barrier function of the body (protective function against pathogen infection via mucosa), and to maintain or improve antiviral function.

Specifically, the composition of the present invention can be used as an antiviral composition.

### (Young Barley Grass)

Barley (Hordeum vulgare L.) is said to be native of Central Asia, is a poaceous annual or biennial grass plant, and is broadly divided into two-row barley, six-row barley, etc. from their spike form. Barley comprises barley β-glucan which is a water soluble dietary fiber, and non-soluble dietary fiber in abundance. Young barley grass used in the composition of the present invention is not particularly limited as long as it is one that is commonly available, and any type of young barley grass such as two-row barley, six-row barley, etc. can be used. Further, it can comprise stem together with young leaves.

As for young barley grass, for example, ground product, juice, extract, etc. can be used. As for ground product, dried powder (young barley grass powder), finely divided powder and its dried product (dried finely divided powder), etc. can be exemplified. Juice or extract can be in liquid form, but can be also used in paste form or as dried powder (juice powder, extract powder). Extract can be obtained by extracting using an appropriate solvent, and examples of solvents include water (warm water, hot water), ethanol, and water-containing ethanol. In the present invention, since it comprises more dietary fiber, and can further enjoy the effect of the present invention, ground product is preferable, and among ground products, dried powder is particularly preferable.

Further, from the viewpoint that young barley grass used in the present invention can further enjoy the effect of the present invention, it is preferable to comprise 20% by mass or more of dietary fiber, more preferably to comprise 25% by mass or more, and further preferably to comprise 30% by mass or more. The upper limit can be for example 70% by mass.

### (Composition of the Present Invention)

The composition of the present invention can be used for example, as medicine (including quasi-drug), or as an oral composition such as so-called health food products such as functional foods which indication of efficacy is allowed from a prescribed authority, including foods for specified health use, foods with nutrient function claims, foods with function claims, or the like.

The composition for increasing regulatory T cells of the present invention is not particularly limited as long as it can be distinguished from other products as product, in the point of comprising young barley grass and of being used for increasing regulatory T cells, and for example those with an indication of a function of increasing regulatory T cells on any of the main body, package, instructions, advertisement (advertising medium) of the product of the present invention are encompassed in the scope of the present invention. Specifically, indications comprising the phrase “increasing Treg cells”, “enhancing action of Treg cells” such as “increase Treg cells, and maintain normal immune function” can be exemplified. Further, in the foods for function claims, those having increase of regulatory T cells as scientific basis of the function are encompassed.

The composition for increasing regulatory T cells of the present invention can be one indicating young barley grass as active ingredients, but is not limited to those with an indication of comprising young barley grass as active ingredients on the package of the product, etc. For example, it can be one not specifying the active ingredients. Further, even it is a general food product, those manufactured and sold by suggesting the use are encompassed in the scope of the present invention. For example, food products etc. sold by posting a personal experience referring to increase of regulatory T cells as a personal impression of an individual having intake the product, on the website, etc. are also encompassed in the scope of the invention.

The composition for suppressing immune response of the present invention is not particularly limited as long as it can be distinguished from other products as product, in the point of comprising young barley grass and of being used for suppressing immune response function, and for example those with an indication of a function of suppressing immune response function on any of the main body, package, instructions, advertisement (advertising medium) of the product of the present invention are encompassed in the scope of the present invention.

Specifically, in a so-called health foods, those indicating “suppressing immune response”, “help to suppress immune response”, “useful to suppress immune response”, “maintain immune function to be normal”, “help to maintain immune function to be normal”, “useful to maintain immune function to be normal”, “maintain normal immune function”, “help to maintain normal immune function”, “useful to maintain normal immune function”, “maintain immunologic homeostasis”, “help to maintain immunologic homeostasis”, “useful to maintain immunologic homeostasis”, “maintain normal mucosal immune function”, “help to maintain normal mucosal immune function”, “useful to maintain normal immune function”, and “useful to maintain normal mucosal immune function” etc. can be exemplified.

The composition for suppressing immune response of the present invention can be one indicating young barley grass as active ingredients, but is not limited to those with an indication of comprising young barley grass as active ingredients on the package of the product, etc. For example, it can be one not specifying the active ingredients. Further, even it is a general food product, those manufactured and sold by suggesting the use are encompassed in the scope of the present invention. For example, food products sold by posting a personal experience referring to suppression of immune response as a personal impression of an individual having intake the product, on the website, etc. are also encompassed in the scope of the invention.

Further, the composition for maintaining normal immune function of the present invention is not particularly limited as long as it can be distinguished from other products as product, in the point of comprising young barley grass and of being used for maintaining normal immune function, and for example those with an indication of a function of maintaining normal immune function on any of the main body, package, instructions, advertisement (advertising medium) of the product of the present invention are encompassed in the scope of the present invention.

Specifically, in a so-called health foods, those indicating “suppressing immune response”, “help to suppress immune response”, “useful to suppress immune response”, “maintain immune function to be normal”, “help to maintain immune function to be normal”, “useful to maintain immune function to be normal”, “maintain normal immune function”, “help to maintain normal immune function”, “useful to maintain immune function”, “maintain immunologic homeostasis”, “help to maintain immunologic homeostasis”, “useful to maintain immunologic homeostasis”, “maintain normal mucosal immune function”, “help to maintain normal mucosal immune function”, “useful to maintain normal immune function” and “useful to maintain normal mucosal immune function” etc. can be exemplified.

The composition for maintaining normal immune function of the present invention can be one indicating young barley grass as active ingredients, but is not limited to those with an indication of comprising young barley grass as active ingredients on the package of the product, etc. For example, it can be one not specifying the active ingredients. Further, even it is a general food product, those manufactured and sold by suggesting the use are encompassed in the scope of the present invention. For example, food products sold by posting a personal experience referring to maintenance of normal immune function as a personal impression of an individual having intake the product, on the website, etc. are also encompassed in the scope of the invention.

Further, the antiviral composition of the present invention is not particularly limited as long as it can be distinguished from other products as product, in the point of comprising young barley grass and of being used for anti-virus (protecting from viral infection), and for example those with an indication of an antiviral function on any of the main body, package, instructions, advertisement (advertising medium) of the product of the present invention are encompassed in the scope of the present invention.

Specifically, in so-called health foods, those indicating “maintain barrier function of the body”, “help to maintain barrier function of the body”, “elevating barrier function of the body”, “help to elevate barrier function of the body”, “increasing barrier function of the body”, “help to increase barrier function of the body”, “enhancing barrier function of the body”, “help to enhance barrier function of the body”, “maintain protection of the body”, “hep to maintain protection of the body”, “elevating protection of the body”, “hep to elevate protection of the body”, “maintain barrier function of mucosa”, “help to maintain barrier function of mucosa”, “elevate barrier function of mucosa”, “help to elevate protection of mucosa”, “maintain protection of mucosa”, “help to maintain protection of mucosa”, “elevate protection of mucosa”, “help to elevate protection of mucosa”, “maintain protection against virus”, “help to maintain protection against virus”, “elevate protection against virus”, “help to elevate protection against virus”, “enhancing protection against virus”, “help to enhance protection against virus”, “maintain normal immune function”, and “help to maintain normal immune function” etc. can be exemplified.

The antiviral composition of the present invention can be one indicating young barley grass as active ingredients, but is not limited to those with an indication of comprising young barley grass as active ingredients on the package of the product, etc. For example, it can be one not specifying the active ingredients. Further, even it is a general food product, those manufactured and sold by suggesting the use are encompassed in the scope of the present invention. For example, food products sold by posting a personal experience referring to antiviral function as a personal impression of an individual having intake the product, on the website, etc. are also encompassed in the scope of the invention.

Examples of the form of the composition of the present invention include, for example, tablet form, capsule form, powder form, granule form, liquid form, grain form, bar form, plate form, block form, solid form, pellet form, paste form, cream form, caplet form, gel form, chewable tablet form, stick form, or the like. It can be in form of ice cream, jelly, cookies, cake, chocolate, PET bottle beverage, etc. Among these, the form of tablet form, capsule form, powder form, granule form, pellet form, chewable tablet form is preferable, and powder form and granule form are particularly preferable.

In case the composition of the present invention is made into a tablet form, pellet form, chewable tablet form, it is preferable to add one or more of any of excipient, lubricant, or fluidizer. As such, the molding property is enhanced, and the storage stability of the produced agent is improved. Particularly, by using an excipient and lubricant, the storage stability can be further improved.

As for the content of young barley grass in the composition of the present invention, it can be comprised within the scope with which the effect is exerted. For example, in the composition of the present invention, in dry mass equivalent, young barley grass can be comprised in an amount of 0.001 to 100% by mass, and it is preferable to be comprised in an amount of 0.01 to 100% by mass, more preferable to be comprised in an amount of 0.1 to 100% by mass, and further preferable to be comprised in an amount of 1 to 100% by mass.

The amount of intake of young barley grass in the present invention is not particularly limited. However, to further enjoy the effect more effectively, it is preferable to intake so that the amount of intake of young barley grass of an adult per day is 0.25 g or more, more preferable to intake so that it is 0.5 g or more, and further preferable to intake so that it is 1.0 g or more. The upper limit is for example 30 g, preferably 20 g, and more preferably 10 g.

The composition of the present invention can be appropriately designed so that the amount of intake per day of young barley grass becomes the above-mentioned amount of intake, and it can be an embodiment to be taken at once, or an embodiment to be taken in plural times. Specifically, for example, it can be stored as an amount per day in one container, or for example in plural containers of two to four.

The composition of the present invention can be produced by known methods, by using young barley grass. Further, it can be produced by known method, by adding ingredients other than young barley grass, according to need.

### EXAMPLES

In the following, the present invention will be explained in further details by the Examples. However, the present invention is not limited to these Examples.

### Example 1

The influence of intaking young barley grass on regulatory T cells (Treg cells) has been investigated.

### <Sample>

As raw material, aerial part of barley (leaf and stem) harvested at a height of about 30 cm was used. This was subjected to pretreatment by washing with water to remove attached soil, etc. and cut to a size of about 5 to 10 cm. Pretreated stems and leaves were subjected to blanching treatment once with boiling water of 90° C. to 100° C. for 90 sec to 120 sec, and then cooled down with cold water. Then, the obtained stems and leaves were dried in a drier for 20 min to 180 min, with hot air of 80° C. to 130° C. until the water content becomes 5% by mass or less. The dried stems and leaves were subjected to coarse crushing treatment to a size of about 1 mm. By subjecting the obtained stems and leaves of barley to fine crushing treatment, dried powder of young barley grass (young barley grass powder) was produced.

### <Test Method>

Male ICR mice of 4 weeks-old were acclimated for 2 weeks. At the age of 6 weeks-old, mice were classified into three groups based on the body weight (each group n=10), and were allowed to intake NFD (non fiber dietary), CLD (cellulose added dietary) or YBD (young barley grass powder (YBLP) mixed dietary) for 3 weeks, respectively. The details of the composition of the feedstuff are shown in Table 1. During the test period, mice were allowed to freely drink water and freely eating.

TABLE 1

| Ingredient | NFD | CLD | YBD |
|---|---|---|---|
| Casein | 20 | 20 | 20 |
| β Corn Starch | 44.7486 | 39.7486 | 31.5486 |
| α Corn Starch | 13.2 | 13.2 | 13.2 |
| sucrose | 10 | 10 | 10 |
| Soybean oil | 7 | 7 | 7 |
| Mineral Mix | 3.5 | 3.5 | 3.5 |
| (AIN-93G) | | | |
| Vitamin Mix | 1 | 1 | 1 |
| (AIN-93) | | | |
| L-Cystine | 0.3 | 0.3 | 0.3 |
| Choline Bitartrate | 0.25 | 0.25 | 0.25 |
| t-Butylhydroquinone | 0.0014 | 0.0014 | 0.0014 |
| Cellulose | — | 5 | — |
| YBLP | — | — | 13.2 |
| Total (%) | 100 | 100 | 100 |

After three weeks, Peyer's patch was extracted from mouse small intestine, put in 5% FBS containing RPMI 1600 medium, and the Peyer's patch was crushed with a 70 mm nylon mesh, to collect cells. FoxP3 positive T cells ratio (Treg ratio) in the total CD4 positive cells was analyzed with a flow cytometer.

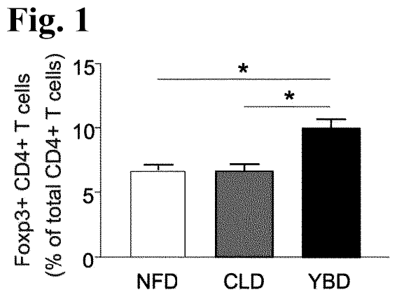

FIG. 1 shows the results of Treg cells ratio.

As shown in FIG. 1, in NFD intake group, it was 6.78±0.41%, in CLD intake group 6.70±0.52%, while in the YBD intake group of the present invention, it was 10.00±0.699%. Specifically, it was confirmed that Treg cells ratio was significantly high in the YBD intake group of the present invention.

Therefore, the composition comprising young barley grass of the present invention has an effect of increasing Treg cells, and can suppress immune response to maintain normal immune function.

### Example 2

### (Production of Powder Beverage)

Powder beverage consisting of the following ingredients was produced. The powder beverage exerted the effect of the present invention.

| | | |
|---|---|---|
| Young barley grass powder | 74% | by mass |
| Green tea | 4% | by mass |
| Maltitol | 22% | by mass |

### Example 3

### (Production of Cookies)

Cookies consisting of the following ingredients were produced. The cookies exerted the effect of the present invention.

| | | |
|---|---|---|
| Young barley grass powder | 7.41% | by mass |
| Wheat powder | 37.04% | by mass |
| Butter | 15.43% | by mass |
| Sugar | 18.52% | by mass |
| Baking powder | 1.54% | by mass |
| Egg | 20.06% | by mass |

### INDUSTRIAL APPLICABILITY

Since the composition of the present invention can be used as health food products, etc., it is industrially applicable.

1-4. (canceled)

5. A method for increasing regulatory T cells, comprising administering a composition comprising young barley grass to human.

6. A method for suppressing immune response, comprising administering a composition comprising young barley grass to human.

7. A method for maintaining normal immune function, comprising administering a composition comprising young barley grass to human.

8. A method for maintaining or improving antiviral function, comprising administering a composition comprising young barley grass to human.
